(19) United States
(12) Patent Application Publication
(10) Pub. No.: US 2023/0354932 A1
(43) Pub. Date: Nov. 9, 2023

(54) Body Carrier System for Mobile Device

(71) Applicant: CORTEX Biophysik GmbH, Leipzig (DE)

(72) Inventors: Andreas Günther, Leipzig (DE); Markus Siepmann, Mulheim an der Ruhr (DE)

(21) Appl. No.: 18/142,754

(22) Filed: May 3, 2023

(30) Foreign Application Priority Data
May 4, 2022 (DE) 10 2022 111 06

(51) Int. Cl. A41D 13/12

(52) U.S. Cl. CPC A41D 13/1281; A41D 13/1245

(57) ABSTRACT

The present invention relates to a body carrier system for a mobile device, comprising
a vest-like carrying element, a back part, two shoulder straps connected to the back part and a hip belt connected to the back part, and
a two-part holding element for the mobile measuring device, whose both sub-elements can be reversibly fixed to the vest-like carrying element, as well as reversibly connected to each other,
   wherein each shoulder strap is equipped with welts in the front and/or
   wherein spaced welts are provided on the back part of the vest-like carrying element,
   and wherein on each of the two sub-elements of the two-part holding element welt guides are provided, so that the welt guides can be engaged with the welts.

The invention deals with a body carrier system for a mobile device, which can be used, in particular, in spiroergometry.

In the diagnostic procedure of spiroergometry (also known as ergospirometry or ergospirography), heart reactions, blood circulation, breathing, metabolism, and/or cardiopulmonary performance are qualitatively and quantitatively examined by measuring respiratory gases during physical stress. This examination is not only interesting in stationary conditions, e.g. at a doctor’s surgery, but also outdoors, where real stress conditions exist when running, climbing, cycling and the like.

For outdoor mobile spiroergometry, however, a suitable portable device must be used, which, first of all, should be of a small size and, therefore, of a small weight, so that it causes no disturbance during physical stress, or only to a very small extent. Such devices are generally known, but often have a very limited range of use, due to the above-mentioned requirements.

During mobile outdoor applications, strong and sometimes very different acceleration forces act on a portable device, depending on the type of application (running, cycling, paddling, etc.). Accordingly, the carrying system and the device attachment worn by the subject must be capable of absorbing such forces without having an uncomfortable and thus constricting effect. In addition, it is necessary for the various types of applications that the portable device can be flexibly positioned on the body in order to determine the course of movement depending on the sports type or physical stress.

For carrying a portable device, previous state-of-the-art approaches provide, for example, carrying bags, which are, however, quite inflexible when it comes to positioning and uncomfortable when stowing the portable devices, electrical cables or pneumatic pipes. Alternatives include rigid metal scaffolds, which are even less flexible in positioning the portable device and can be carried almost exclusively on the back. In addition, these alternatives require a great deal of effort when fixing them (e.g. by means of bolted connections).

In view of the state of the art described above, there is a need for a flexible solution for carrying portable devices.

The purpose of the present invention is therefore to provide a novel body carrier system for mobile devices, which is flexible in handling, which allows a flexible positioning of the mobile device, which can be used for activities of various intensity without disturbing, and which allows a mobile device to be carried safely on the body.

In the present invention, this purpose is achieved by a body carrier system (5) for a mobile device (1), comprising
a vest-like carrying element (51), comprising a back part (511), two shoulder straps (53a, 53b) connected to the back part (511) and a hip belt (55) connected to the back part (51), and
a two-part holding element (7) for the mobile measuring device, whose both sub-elements (7a, 7b) can be reversibly fixed to the vest-like carrying element (51), as well as reversibly connected to each other,
wherein each shoulder strap (53a, 53b) is equipped with welts (57) in the front and/or
wherein spaced welts (57) are provided on the back part (511) of the vest-like carrying element (51),
and wherein on each of the two sub-elements (7a, 7b) of the two-part holding element (7) welt guides are provided (71a, 71b), so that the welt guides (71a, 71b) can be engaged with the welts (57).

For the purposes of the present invention, the term “vest-like carrying element” refers to a specially designed waistcoat which can be put on like such. In addition, this specially designed waistcoat includes the other elements to be explained below.

The term “welt” is generally known and usually refers to the edge reinforcement of a cloth or tarpaulin. The welt usually has a round diameter and is designed to hold a cloth applied as what is known as a “welt flag” in a welt guide and thus establish a connection.

In the inventive body carrier system, (5) the welts (57) are tightly and firmly connected to the vest-like carrying element (51), unlike in the usual use of welts.

In the present invention the “welt guides” are designed similarly to the known welt guides and can be pushed onto the welts. In the present invention, the sub-elements (7a, 7b) of the two-part holding element (7) provided with the welt guides (71a, 71b) are movable in relation to the inventive the body carrier system (5).

The present invention generally has the advantage that the inventive body carrier system (5) is a kind of functional garment, which ensures a flexible and, at the same time, secure attachment of the mobile device (1) for physical activities of various intensity. The mobile device (1) can thus be safely carried on the body without significantly disturbing or obstructing the carrier. The welts (57) provided on the front and rear of the inventive body carrier system (5), moreover, increase the invention variability with regard to the mobile device positioning.

Minimalist and sophisticated design and smartly integrated features give the subject the possibility to put on and remove the inventive body carrier system (5) in the shortest possible time independently and without external help. In addition, the inventive body carrier system (5) can be offered in different clothing sizes.

In addition, the inventive body carrier system (5) has a very light weight of about 600 g (slightly depending on the actual clothing size) and does not pose a significant weight load on the subject.

A particularly preferred feature of the welt guides (71a, 71b) is that their inner profile is conical. The larger cone diameter facilitates threading onto the welt (57), while the smaller cone diameter on the other side ensures increased friction between the welt (57) and the welt guide (71a, 71b), so that the “slip resistance” is increased.

In an advantageous further development, the welt guides (71a, 71b) of the two sub-elements (7a, 7b) of the two-part holding element (7) are positioned connected to the vest-like supporting element (51) at a tilted angle to the welts (57).

In other words, in this particularly preferred feature of the welt guides (71a, 71b), their axis is tilted relative to the central axis of the two-part holding element (7) by an angle greater than 0° to 15°. This increases the friction between the welt (57) and the welt guide (71a, 71b) and thus the “slip resistance”.

The feature combinations of welt (57) and welt guides (71a, 71b) enable the two-part holding element (7) to be infinitely adjustable in height and thus the mobile device (1) along the welts (57). The welts can therefore be optimally positioned depending on the application area, type of sport, body size, etc.

In a preferred embodiment of the inventive body carrier system (5) the two sub-elements (7a, 7b) of the two-part holding element (7) are positively (form-closed) connectable by means of a hook connection (73) and form a fixed casing for the mobile device (1).

In particular, the hook connection (73) is closed by a movement away from the body of the carrier and then locked, i.e. a solid casing is formed. If the mobile device (1) is used, the hook connection (73) cannot be unfastened — neither intentionally nor unintentionally. This two-part design of the holding element (7) also has the advantage that the sub-elements (7a, 7b) can be threaded on the welts (57) more easily than would be the case with a one-part holding element.

In another embodiment the welt (57) and the two-part holding element (7) are arranged in such a way that the mobile device (1) can be positioned axis-centred (toward the body) and / or close to the body.

The feature combinations lead to the advantage that, initially, by the axis-centred positioning due to the weight of the mobile device (1), any weight asymmetry is avoided which may hinder the carrier in his motions. The close-to-the-body positioning paired with the very low installation height of the two-part holding element (7) leads to only minimal torques, even during strong movements. This also reduces obstacles during movements and minimizes the risk of slipping of the mobile device (1).

Since many types of sport in which the inventive body carrier system (5) is used are very movement-intensive, it turned out to be advantageous that the vest-like carrying element (51), moreover, includes a closable chest strap (59) provided at the height of the welt (57). This ensures that the shoulder straps (53a, 53b) do not slip down from your shoulders during strong movements.

Another feature that additionally contributes to the good and tight fit of the inventive body carrier system (5) is that the hip belt (55) is provided with an adjustable belt buckle (551) to be closed with a magnetic closure system. The magnetic closure system has the additional advantage that it can be opened easily, also with one hand.

Finally, it turned out to be very positive for the firm fit and comfort of the inventive body carrier system (5) that the shoulder straps (53a, 53b) are fastened via an adjustable and/or elastic webbing (531) at the ends of the hip belt (55).

For example, if the mobile device (1) is designed for spiroergometry, cables and hoses are necessary for connecting a breathing mask and other components. To stow and to secure them, fastening lugs (535) are preferably provided on the shoulder straps (53a, 53b) to keep the cables and hoses of the mobile device (1). This also prevents kinking of the hoses. Since the fastening lugs (535) are preferably provided with a magnetic closure, a subject can easily release them with one hand.

In another further development, the back part (511) has an arrangement of cushions and net elements and, moreover, at least one pocket (513) to hold various objects. Cushions and net elements increase the wearing comfort of the inventive body carrier system (5). The integrated pocket can keep, for example, spare batteries for the mobile device (1) or excess lengths of cables and hoses.

The second aspect of the present invention relates to a mobile system for detecting respiratory gas components, comprising
a body carrier system (5) as described above, and
a mobile device (1)
wherein the mobile system is designed for mobile performance diagnostics in spiroergometry.

Due to the inventive two-part holding element (7) provided in the inventive body carrier system (5), the mobile device (1) can be attached and removed very quickly and easily.

As stated above, the inventive body carrier system (5) weighs about 600 g, while the mobile device (1) (including batteries) weighs about 580 g. The mobile system for detection of respiratory gas components has a total weight of only around 1,200 g.

Further objectives, features, advantages and possible applications are derived from the following description of examples that do not restrict the invention, also with reference to the figures. In this case, all the described and/or pictorially illustrated features, in isolation or in any combination, constitute the subject matter of the invention, even irrespective of their summary in the claims or their referring. Illustrations show, as follows:

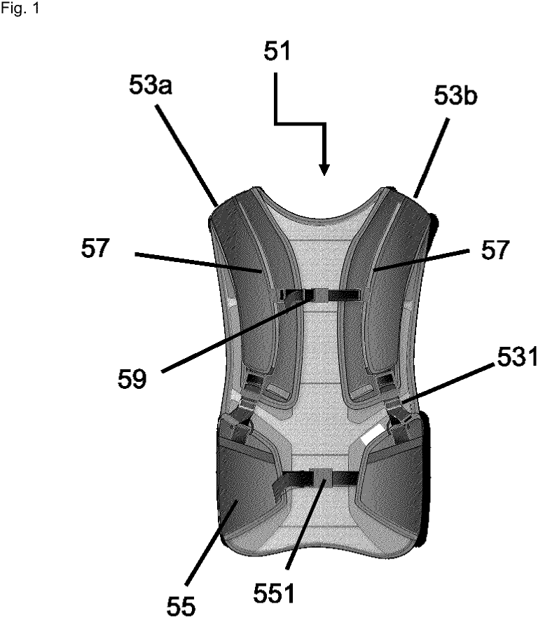

FIG. 1 a graphical representation of the front of a vest-like carrying element 51 according to a preferred example of the invention,

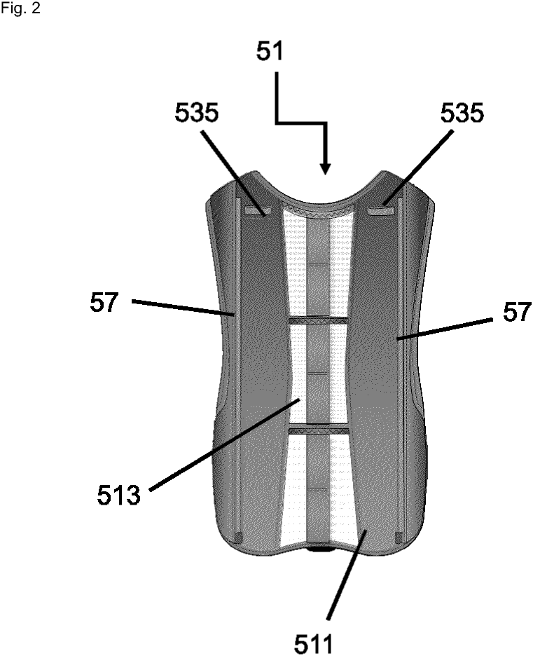

FIG. 2 a graphic representation of the rear side 511 of a vest-like carrying element 51 according to a preferred embodiment of the invention

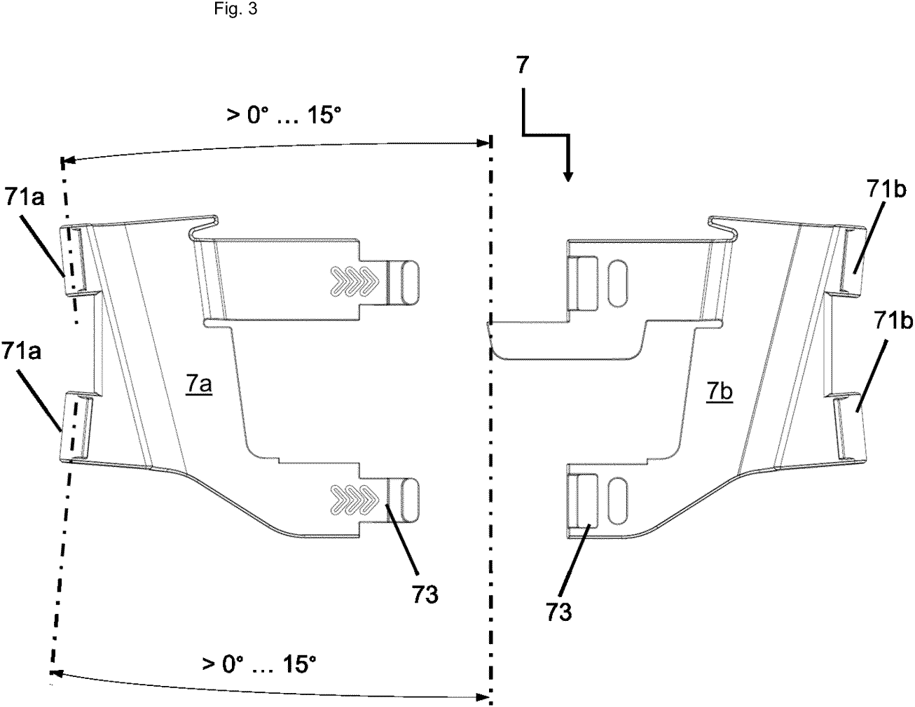

FIG. 3 a graphical representation of the two-part holding element 7 according to a preferred embodiment of the invention,

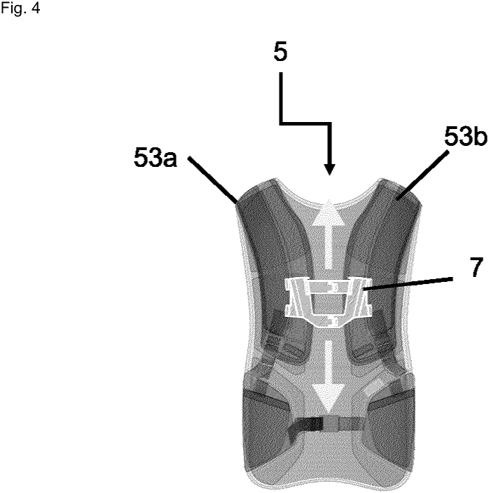

FIG. 4 a schematic representation of the inventive body carrier system 5 according to a preferred embodiment of the invention with a front-mounted two-part holding element 7,

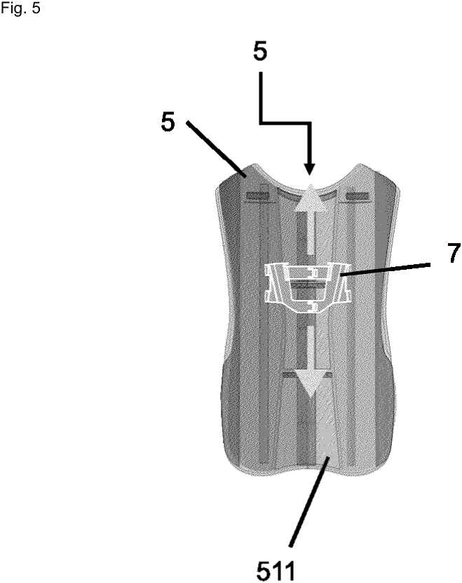

FIG. 5 a schematic representation of the inventive body carrier system 5 according to a preferred embodiment of the invention with a two-part holding element 7 arranged on the back,

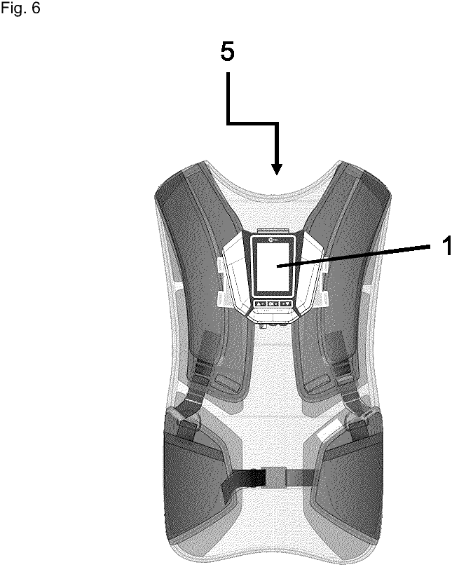

FIG. 6 a schematic representation of a mobile system for detecting respiratory gas components, according to a preferred embodiment of the invention, with inventive body worn system 5 and mobile device 1 attached thereto, and

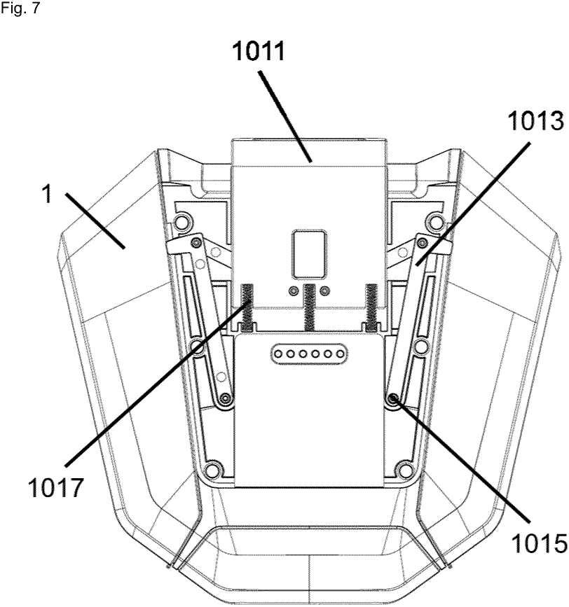

FIG. 7 a graphic representation of the locking system on the mobile device 1 according to an embodiment of the invention.

In the figures, all the same components are named with the same reference marks, but for more clarity, not all reference marks are necessarily inserted in all representations.

FIG. 1 shows a graphical representation of the front of a vest-like carrying element 51 according to a preferred embodiment of the invention, in which each of the twoshoulder carriers 53a, 53b are provided with a welt 57. Each shoulder strap 53a, 53b is fastened with an elastic webbing 531 at the ends of the hip belt 55, which is closable by an adjustable belt buckle 551.

The rear side 511 of the vest-like carrying element 51 is shown schematically in FIG. 2. In this embodiment, two welts 57 are arranged at some distance to each other. Between them there is a pocket 513. In the upper area of the rear part 511, fastening lugs 535 are provided for keeping cables and hoses.

FIG. 3 shows a detailed graphical representation of the two-part holding element 7 according to a preferred embodiment of the invention. Its function is to be described below in more detail. In this illustration, two welt guides 71a, 71b are provided for each of the two sub-elements 7a, 7b. The welt guides can be threaded onto the welts 57. In this illustration, the tilted angle of the welt guides 71a, 71b relative to the central axis can be clearly seen; it is slightly more than 0° to 15°.

FIG. 4 schematically illustrates how the two-part holding element 7 is arranged on the front side of the inventive body worn system 5 according to a preferred embodiment of the invention. The arrows indicate how the holding element 7 can be positioned flexibly in height.

Similarly, FIG. 5 schematically shows how in the inventive body carrier system 5, according to a preferred embodiment, the two-part holding element 7 is arranged on the front, wherein the arrows indicate how the holding element 7 can be positioned flexibly in height also on the back.

A schematic representation of a mobile system for detecting respiratory gas components, according to a preferred invention embodiment, is shown in FIG. 6. In this illustration, a mobile device 1 is attached to the inventive body carrier system 5 on the front, i.e. on the chest of the subject by means of a holding element 7.

Finally, FIG. 7 schematically shows how the locking system on the mobile device 1 is shaped, according to an embodiment of the invention,, so that it can also be mounted and removed with one hand.

A preferred embodiment, but not limiting the present invention, is described below.

In conjunction with the development of a novel system for the detection of respiratory gas components, which can be used in particular in spiroergometry, the need for a new functional carrying system for subjects was determined that, paired with the newly designed mobile device 1, can facilitate spiroergometric measurements or performance diagnostics in mobile applications (e.g. field tests or laboratories).

The new carrying system should offer maximum comfort and freedom of movement for the subject and meet all requirements of mobile performance diagnostics, regardless of the specific application area. Another particular focus was on the intuitive and simple use of the carrying system in interaction (simple integration) with the mobile device 1, when taking into account an optimal weight distribution for the subject and flexible positioning of the mobile device 1 on the subject’s body.

The inventive body carrier system 5 according to this particular embodiment shown in the figures has two essential functional individual elements on the front (breast part), the shoulder straps 53a, 53b and the hip belt 55. Optionally, a chest strap 59 can be provided.

Compared to support systems known in spiroergometry to date, the ergonomically shaped shoulder straps 53a, 53b improve the subject’s freedom of movement and the wearing comfort. The shoulder straps are preferably connected to an elastic webbing strap 531 (including D-ring) at the ends of the hip belt 55 on the left and right, and can be adjusted lengthwise for better alignment to the subject’s body size. This ensures that the shoulder pads of shoulder straps 53a, 53b are ergonomically resting on the shoulders. The upper area of the shoulder straps contains fastening lugs 535 for the cables and hoses of the gas extraction section or the volume flow sensor. In particular, they are magnetic loops that can be easily fastened and detached by the subject himself. These flexible appliances prevent the cables/hoses from kinking/crushing, so that excess cable lengths can be directly stowed and individually adjusted depending on whether the mobile device 1 is placed on the chest or back.

As an essential functional element, each of both shoulder straps is provided with a welt 57. The welts are not only intended for additional shaping of the shoulder carriers 53a, 53b, but mainly for fastening the two-part holding element 7 of the mobile device 1. They allow for almost free vertical positioning in the chest area.

The multi-layer padded, flexible and anatomically shaped hip belt 55 provides a firm and comfortable fit on the pelvic bones and transfers the entire weight of the mobile device 1 to the hips during the movement-intensive sport or measurement process. The hip belt 55 is preferably provided with a Fidlock belt buckle (adjustable belt buckle 551), which works with a magnetic closure system to ensure a quick application or to enable the subject to put on the inventive body carrier system 5 himself.

The (optional) chest straps 59 that can be fixed to the welts 57 are adjustable in height and width and prevent the shoulder straps 53a, 53b from slipping sideways. In conjunction with the mobile device 1, a better wearing comfort is thus ensured, taking into account an even weight distribution (centre of gravity close to the centre of gravity of the subject’s body).

The seamlessly stitched back part 511 and the special placement of foam and mesh cushions optimize pressure distribution and also ventilation for maximum comfort. In addition, three additional pockets with clamps are integrated on the rear side of the inventive body carrier system 5 to easily and quickly put equipment (e.g. smartphones, etc.) into them.

Furthermore, on the rear side of the inventive body carrier system 5 there are also welts 57 that enable a safe and flexible positioning of the mobile device 1 also on the back part 511 with the aid of the specially designed two-part holding element 7 .

An essential requirement for the new carrying system was optimal positioning of the mobile device 1 during mobile performance diagnostics. In this case, the mobile device 1 should always be optimally attached to the subject without restricting his freedom of movement. In addition, it was important to consider that the centre of gravity of the mobile device 1 is always close to the subject’s body (both in the chest and back area) in order to achieve better control over occurring acceleration forces. To meet these requirements, a specially designed two-part holding element 7 was developed. It consists of a left and a right sub-element 7a, 7b and can be understood as a kind of casing in such a composite form.

In order to optimally integrate the two-part holding element 7 into the inventive body carrier system 5, the respective sub-element 7a, 7b provided with a special welt guide 71a, 71b must first be a) inserted on the left and right shoulder strap 53a, 53b or b) pushed into the back part 511 of the inventive body carrier system 5 on the left and right side. After that both sub-elements 7a, 7bshould be connected positively (form-closed) with a hook connection 73. The holding element 7 designed as a casing guarantees that there are no pressure points on the subject’s body. Rigidity of the holding element 7 combined with the welts 57 bent in the chest area and separated in the back area generates tensile stress, which prevents slipping. In addition, the two welt guides 71a, 71b, each located on the right and left sub-element 7a, 7b of the holding element 7, are arranged at a certain angle, whereby an additional frictional force is generated, which prevent slipping even at larger forces.

The mobile device 1 has a special locking system at the rear, with the aid of which the mobile device a) is locked in a stationary device 3 (docking station) and b) can be fixed in the holding element 7 of the inventive body carrier system 5. This interlocking system is shown schematically in FIG. 7. The subject can trigger the snap-in function himself by one hand. To do so, he has to press down the “CLICK & GO” button 1011 on the mobile device 1 with his index and middle finger. As a result, the lateral locking hooks 1013 are pulled inwards via their pivot points 1015 and the locking mechanism is released (release function). The mobile device 1 can then be placed in the casing of the holding element 7. As soon as the subject releases the “CLICK & GO” button, the locking hooks 1013 and “CLICK & GO” button 1011 are returned to the initial position (lock function) via a spring mechanism 1017. This functionality ensures that the mobile device 1 remains locked in the inventive body carrier system 5 even in the event of possible external influence.

Reference marks list

| | |
|---|---|
| 1 | mobile device |
| 1011 | “CLICK & GO” button |
| 1013 | snap-fit hook |
| 1015 | pivot point |
| 1017 | spring mechanism |
| 5 | body carrier system |
| 51 | vest-like carrying element |
| 511 | back panel |
| 513 | bag |
| 53a, 53b | shoulder strap |
| 531 | elastic belt strap |
| 535 | fastening lug |
| 55 | hip belt |
| 551 | belt buckle |
| 57 | welt |
| 59 | chest strap |
| 7 | holding element |
| 7a, 7b | sub-elements |
| 71a, 71b | welt guides |
| 73 | hook connection |

1. Body carrier system for a mobile device comprising
a vest-like carrying element, comprising a back part, two shoulder straps connected to the back part and a hip belt connected to the back part, and
a two-part holding element for the mobile measuring device, whose both sub-elements can be reversibly fixed to the vest-like carrying element, as well as reversibly connected to each other,
wherein each shoulder strap is equipped with welts in the front and/or
wherein spaced welts are provided on the back part of the vest-like carrying element,
and wherein on each of the two sub-elements of the two-part holding element welt guides are provided, so that the welt guides can be engaged with the welts.

2. Body carrier system according to claim 1, wherein the welt guides of the two sub-elements of the two-part holding element are at a tilted angle to the welts in the position connected to the vest-like supporting element.

3. Body carrier system according to claim 1, wherein the two sub-elements of the two-part support element are positively (form-closed) connectable by means of a hook connection and form a fixed casing for the mobile device.

4. Body carrier system according to claim 1, wherein the welt and the two-part support element are arranged in such a way that the mobile device can be positioned axis-centred (toward the body) and / or close to the body.

5. Body carrier system according to claim 1, wherein the vest-like carrying element, moreover, includes a closable chest strap provided at the height of the welt.

6. Body carrier system according to claim 1, wherein the open end of hip belt is provided with an adjustable belt buckle to be closed with a magnetic closure system.

7. Body carrier system according to claim 1, wherein each of the two shoulder straps is fastened by an adjustable and/or elastic webbing at the ends of the hip belt.

8. Body carrier system according to claim 1, wherein on the shoulder carriers fastening lugs are provided for holding cables and hoses of the mobile device.

9. Body carrier system according to claim 1, wherein the back part has an arrangement of cushions and net elements and, moreover, at least one pocket to hold various objects.

10. Mobile system for detection of respiratory gas components, consisting of
a body carrier system according to claim 1 and
a mobile device
wherein the mobile system is designed for mobile performance diagnostics in spiroergometry.
